(19) United States
(12) Patent Application Publication
(10) Pub. No.: US 2024/0404672 A1
(43) Pub. Date: Dec. 5, 2024

(54) PLATFORM FOR IDENTIFYING NOVEL ANALGESIC THERAPIES

(71) Applicant: EPTIVA THERAPEUTICS LTD., Oxford Oxfordshire (GB)

(72) Inventor: Mark J. FIELD, Great Dunmow (GB)

(21) Appl. No.: 18/697,695

(22) PCT Filed: Oct. 8, 2022
(86) PCT No.: PCT/IB2022/059656

§ 371 Date: Apr. 1, 2024

Related U.S. Application Data
(60) Provisional application No. 63/253,619, filed on Oct. 8, 2021.

(51) Int. Cl. G16H 20/10; G16B 5/00; G16H 10/20; G16H 10/60

(52) U.S. Cl. CPC G16H 20/10; G16B 5/00; G16H 10/20; G16H 10/60

(57) ABSTRACT

The present disclosure is directed to a platform for linking therapeutic compounds to pain phenotypes/conditions and thereby identifying potential novel analgesic therapies. The platform can generate patient selection criteria and/or identify a subset of patients that would be suitable for a clinical trial related to the selected therapeutic compound and the one or more identified pain phenotypes/conditions.

### CROSS-REFERENCE TO RELATED APPLICATIONS

This application claims priority to and the benefit of U.S. Provisional Patent Application No. 63/253,619, filed Oct. 8, 2021, the entirety of which is incorporated herein by reference.

### BACKGROUND

### Technical Field

This disclosure relates to a platform for linking therapeutic compounds to pain disorders and using the associations to identify novel analgesic therapies.

### Related Technology

Chronic pain is a burden to many patients. It has been suggested that up to 1 in 4 people will suffer a chronic pain condition, creating associated economic costs estimated at $600 billion every year in the U.S. alone. Despite the intense need for new analgesic therapies, research and development for pain treatment receives relatively little funding due to traditionally low success rates. New analgesic therapies have only a 2% chance of clinical success, one of the lowest rates for any type of therapeutic. As a comparison, other therapeutic areas experience a clinical trial success rate of about 10%.

Conventional strategies for analgesic therapy development include (1) the development of new drugs with novel mechanisms of action, and (2) repurposing of known drugs to address new indications and/or patient populations. Even though the latter strategy does not require the development of novel compounds, the clinical development costs remain very high. The immense costs of these development efforts are due in large part to the inability to identify which compounds are more likely to have clinical success for a particular pain condition. Often, unsuccessful therapies are not determined as such until large clinical trials have already been conducted. There is a high attrition rate in the pain area with 98% of projects failing in clinical development.

Accordingly, there is an ongoing need for methodologies that link potential therapeutic targets to particular pain disorders in a manner that increases the likelihood of clinical success.

### SUMMARY

The present disclosure is directed to a platform for linking therapeutic compounds to pain phenotypes/conditions and thereby identifying potential novel analgesic therapies.

In one embodiment, a computer system comprises one or more processors and one or more hardware storage devices having stored thereon computer-executable instructions which are executable by the one or more processors to cause the system to at least: receive a selected therapeutic compound; generate a protein-protein interaction map for each of one or more parameter settings for the selected therapeutic compound; for each parameter setting generate a set of predicted gene pathways based on the corresponding protein-protein interaction map, and generate a set of pain phenotype associations based on the protein-protein interaction map and the set of predicted gene pathways; combine the sets of pain phenotype associations from each parameter setting; identify one or more pain phenotypes having sufficient overlap across the combined sets of pain phenotype associations as potential treatment targets; and identify the selected therapeutic compound as a potential analgesic for the one or more treatment targets.

Optionally, the platform can further generate patient selection criteria and/or can identify patients that would be suitable for a clinical trial related to the selected therapeutic compound and the one or more identified pain phenotypes (or a pain condition associated with the one or more pain phenotypes). Because the pain phenotypes and/or pain conditions identified by the platform are specifically linked to the selected therapeutic compound, better tailoring the pool of patients to those that also have the identified pain phenotypes and/or pain conditions will better align the selected therapeutic compound and patient pool, and thereby increase the likelihood of a successful clinical trial.

Analgesic clinical development suffers from notoriously low success rates due to the overinclusion of patients with specific subsets of pain phenotypes/conditions and the general difficulty in identifying appropriate patient groups for the selected research compound. The platform described herein beneficially reduces these problems by enabling clinical trials that can better determine whether the selected therapeutic compound is successful as an analgesic.

Moreover, the platform functions to provide identified pain phenotype and/or pain condition outputs without requiring initial filtering or pre-selection of phenotypes and conditions. Stated differently, the platform is disease agnostic and can therefore identify potential treatment targets that may not have been apparent beforehand. Additionally, because the platform can function based on minimal inputs, the potential for user bias is reduced. For example, in at least some embodiments, the minimum required input is the selected therapeutic compound. After receiving the input, the platform can operate to output potential phenotypes and/or conditions without additional input required.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an example computer environment in which the platform for identifying analgesic therapies may be implemented;

FIG. 2 illustrates an example method, which may be implemented by a computer system such as in FIG. 1, for linking a therapeutic compound to a pain condition;

FIG. 3 illustrates an example data flow using the method of FIG. 2;

FIGS. 4A-4D illustrate example outputs using the method of FIG. 2 for the example therapeutic compound calcitonin gene-related peptide (CGRP), showing a protein-protein mapping operation (FIG. 4A), gene ontology operation (FIG. 4B), and a pain phenotype association operation (FIG. 4C) for a particular parameter setting, and showing example output after combining multiple sets/lists of pain phenotype for each parameter setting (FIG. 4D);

FIGS. 5A-5C illustrate example outputs using the method of FIG. 2 for the example compound GPR18, showing a gene ontology operation (FIG. 5A) and a pain condition association operation (FIG. 5B) for a particular parameter setting, and showing example output after combining multiple sets/lists of pain conditions for each parameter setting (FIG. 5C);

FIG. 6A is a model of the molecular pathways involved in pachyonychia congenita (see Cao et al., Gene Expression Profiling in Pachyonychia Congenita Skin, J Dermatol Sci. 2015 March; 77 (3): 156-165) revised to show possible interaction points at which calcitriol can interact; and

FIGS. 6B and 6C are charts summarizing the results of searches (PubMed and ClinicalTrials.gov, respectively) linking vitamin D, cholecalciferol, and/or calcitriol to specific disease conditions.

### DETAILED DESCRIPTION

### Overview

The present disclosure is directed to a platform for linking therapeutic compounds to pain conditions and thereby identifying potential novel analgesic therapies.

Two key issues currently inhibiting innovation with respect to analgesic therapies are (1) the inability to link research compounds to pain disorders where they may be useful as analgesics, and (2) the use of limited patient groups in clinical development and trials. Conventional pain research and development covers only a handful of pain conditions. Generally, there is a preference to run pain clinical trials using a limited group of patients based on existing pathways to regulatory approval. Thus, most pain clinical trials are conducted using a limited number of clinical conditions which have an existing regulatory path to approval.

As a result of such restricted clinical patient populations, therapeutic compounds being researched as potential analgesic therapies tend to be studied against well-established pain conditions rather than linked to the most appropriate pain condition. A better link between the research therapeutic compound and the pain condition would increase the probability of success in the clinical trials. The use of restricted clinical patient populations is likely a key factor in the extremely high attrition rate and lack of innovation in clinical development of analgesic drugs.

As used herein, the term “therapeutic compound” represents any candidate compound that has potential for use in analgesic therapy in one or more pain conditions. The terms “target compound” “research compound,” “research target,” and similar terms are used synonymously with the term “therapeutic compound.”

### Analgesic Therapy Platform

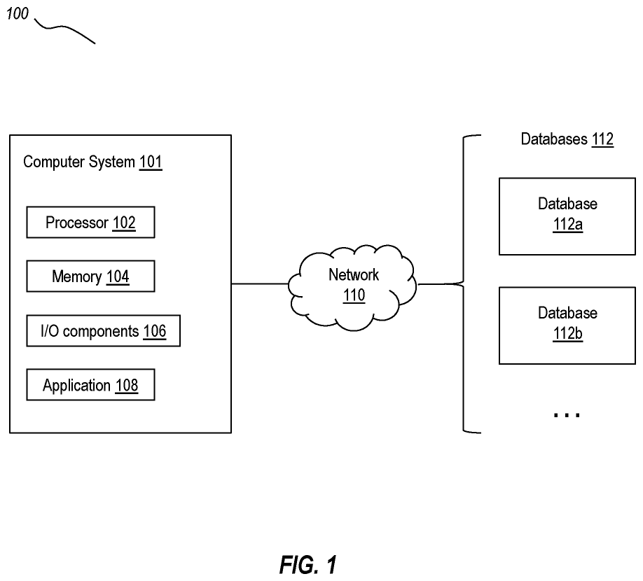

FIG. 1 illustrates an example computer environment 100 in which the platform for identifying analgesic therapies may be implemented. The illustrated computer environment 100 includes a computer system 101 which includes a processor 102 and memory 104. The memory 104 can include physical system memory, which may be volatile, non-volatile, or some combination thereof. The memory 104 may include non-volatile mass storage such as physical storage media (also referred to herein as hardware storage media, computer storage media, computer-readable media, or hardware storage device(s)).

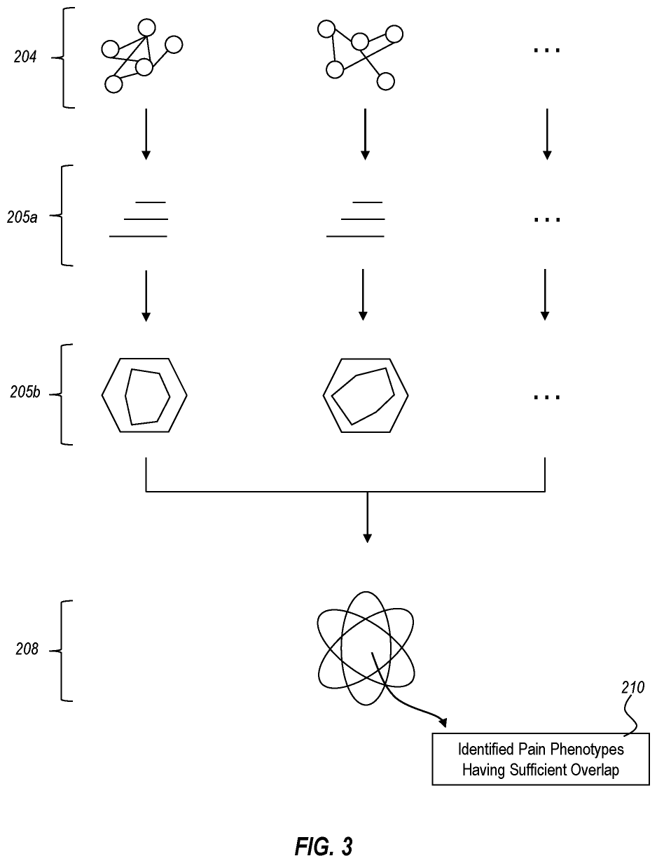

The computer system 101 may also include one or more input/output components 106 such as are known in the art. Examples include keyboards, monitors, touch screens, mouse controllers, speakers, and the like. In some embodiments, the computer system 101 is configured to provide an interface to enable the user to visualize and interact with the various data outputs described herein (e.g., as discussed with respect to FIG. 3).

The computer system 101 may also include an application 108 configured to cause the computer system 101 to implement a method for linking a therapeutic compound to a pain condition, as described in more detail herein.

The computer system 101 may be connected to a network 110. The network 110 may include a cellular network, a Local Area Network (“LAN”), a Wide Area Network (“WAN”), and/or the Internet, for example.

The computer system 101 has access to one or more databases 112 (112a, 112b, etc., where the ellipses indicate that additional databases may be connected and/or accessible). The databases 112 may include: molecular interaction databases such as IntAct, the Molecular INTeraction Database (MINT), MatrixDB, InnateDB, BioGRID, and the Human Protein Reference Database (HPRD), Search Tool for the Retrieval of Interacting Genes/Proteins (STRING); signaling pathway databases such as the Kyoto Encyclopedia of Genes and Genomes (KEGG) and Pathway Commons; gene ontology/transcription factors databases such as JASPAR and Metascape; and human disease/phenotype/condition databases such as Human Phenotype Ontology (HPO), MalaCards, and Open Targets.

The databases 112 may also include a Pain Landscape database that includes 100 or more (e.g., about 300-400) different pain conditions, including rare or genetic conditions that can be addressed through analgesic research and development. This contrasts with the conventional “pain landscape” that has been limited to approximately 10 conditions to address through analgesic research and development. As discussed above, this has contributed to the low research and development success rates for analgesic therapies. As described below, the expanded Pain Landscape database can beneficially enable better matching of therapeutic compounds to particular pain conditions, thereby increasing the likelihood of successful clinical development and implementation.

FIG. 1 represents only one example computing environment. Other embodiments may divide the processing, memory, and/or other functions differently among optional additional computer systems. In some embodiments, memory components and/or program modules are distributed across a plurality of constituent computer systems in a distributed environment. Accordingly, the systems and methods described herein are not limited based on the particular location at which components are located and/or at which functions are performed. Additional details related to the computer environment 100 and/or computer system 101 are provided below.

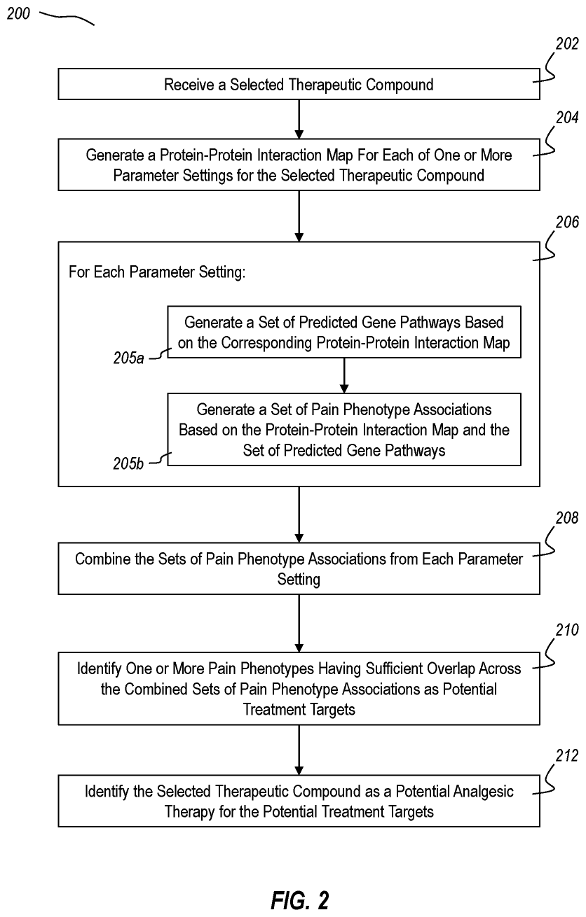

FIG. 2 illustrates an example method 200, which may be implemented by a computer environment/system such as in FIG. 1, for linking a therapeutic compound to a pain condition. As shown, the computer system can receive a selected therapeutic compound (202). The computer system can then operate to generate a protein-protein interaction map for each of one or more parameter settings for the selected therapeutic compound (204). A parameter setting includes the number of connections to analyze and the depth of connectivity to analyze.

For example, a first parameter setting may be set to include 10 first-degree connections and 10 second-degree connections. Under this parameter setting, the protein-protein interaction map would analyze 10 proteins that directly interact (i.e., are first-degree connections) with the selected therapeutic compound and would analyze 10 proteins that interact with the first-degree connections (i.e., are second-degree connections to the selected therapeutic compound). A second parameter setting may be set to include 10 first-degree connections and 20 second-degree connections. A third parameter setting may be set to include 30 first-degree connections and 0 second-degree connections, and so on. Third-degree connections and connections of greater depth can also be included in the operation. Using approximately 4 to 10 different parameter settings has been found to produce effective results, but fewer or more parameter settings may also be utilized.

The protein-protein maps may be generated using a suitable protein-protein database and associated data tools such as described herein. In the specific examples provided below, the STRING database is used. However, other protein-protein interaction databases may additionally or alternatively be utilized.

The computer system may then generate additional data outputs corresponding to each parameter setting (e.g., sequentially and/or in parallel). As shown, the computer system can (206), for each parameter setting, generate a set of predicted gene pathways based on the corresponding protein-protein interaction map (205a) and generate a set of pain phenotype associations based on the protein-protein interaction map and the set of predicted gene pathways (205b).

The set of pain phenotype associations can include symptoms and/or characterizations that are associated with the predicted gene pathways and/or protein-protein interactions. Examples of pain phenotypes include allodynia, chronic lower back pain, migraine disorders, neuralgia, inflammatory pain, skin neoplasms, and the like. Of course, the set of pain phenotype associations will vary based on the particular protein-protein interactions and gene ontology data used as inputs for the pain condition mapping operation. The term “pain condition” is used herein to refer to a condition or disease that is associated with one or more (but typically multiple) pain phenotypes, in contrast to the single pain phenotype itself, though some pain phenotypes (e.g., chronic back pain) can be considered as pain phenotype and as pain conditions themselves.

These operations may be accomplished using a suitable database and associated data tools such as described herein. In the specific examples provided below, the Metascape database is used. However, other gene ontology and/or disease phenotype databases may additionally or alternatively be utilized.

The computer system can then combine/overlay the sets of pain phenotype associations from each parameter setting (208). That is, each parameter setting will likely result in a somewhat different set of pain phenotype associations. These different sets are combined, and the computer system then identifies one or more pain phenotype having sufficient overlap across the combined sets of pain phenotype associations as potential treatment targets (210). Sufficient overlap can be determined based on pre-defined criteria. For example, a pre-defined overlap threshold may require that a pain phenotype be present in at least X % of the sets of pain phenotype associations (e.g., at least 50%, 75%, or 90%). In some embodiments, only those pain phenotypes that are present in each set of pain phenotype associations are identified as potential phenotypes for treatment.

The computer system can then identify the selected therapeutic compound as a potential analgesic therapy for the potential treatment target(s) (212). Optionally, the method may also include the step of linking a particular pain condition from the Pain Landscape database and/or other suitable database to the selected therapeutic compound. For example, step 210 may result in a listing of multiple pain phenotypes that together are associated with a specific pain condition. The Pain Landscape database can include data for multiple pain conditions and their related groupings of pain phenotypes, such that the computer system can operate to identify a pain condition in the Pain Landscape that is associated with the pain phenotypes identified in step 210.

As a specific example described in more detail below, analysis of GPR18 resulted in identification of the pain phenotypes chronic low back pain, tactile allodynia, neuralgia, acute onset pain, and inflammatory pain. These pain phenotypes are together associated with the pain condition radiculopathy. Thus, radiculopathy can be linked to GPR18 as a potential treatment target wherein GPR18 may be utilized as an analgesic therapy.

Optionally, the method 200 can further comprise the step of generating patient selection criteria and/or identifying patients that would be suitable for a clinical trial related to the selected therapeutic compound and the one or more identified pain phenotypes (or pain condition associated with the one or more pain phenotypes). For example, patient information may be stored (e.g., in a patient information database) and the pain phenotypes and/or pain conditions identified by the method can be compared against the patient information and used to filter the patients to select those best matching the set of identified pain phenotypes and/or pain conditions. Stated differently, because the pain phenotypes and/or pain conditions identified by the method are specifically linked to the selected therapeutic compound, better tailoring the pool of patients to those that also have the identified pain phenotypes and/or pain conditions will better align the selected therapeutic compound and patient pool, and thereby increase the likelihood of a meaningful clinical trial.

As discussed above, analgesic clinical development suffers from notoriously low success rates due to the overinclusion of patients with specific subsets of pain phenotypes/conditions and the general difficulty in identifying appropriate patient groups for the selected research compound. The methods described herein beneficially reduce these problems by enabling clinical trials that can better determine whether the selected therapeutic compound is successful as an analgesic. This of course provides several real-world benefits that conventional approaches have failed to achieve.

FIG. 3 illustrates an example data flow using the method of FIG. 2. The reference numbers shown in FIG. 3 correspond to the method steps described in relation to FIG. 2. As related to step 204, a plurality of protein-protein interaction maps are generated, each with a different parameter setting. These maps are shown here as interaction maps with nodes and edges for purposes of illustration, though other data output forms may additionally or alternatively be used. The ellipses indicates that more than two of such protein-protein interaction maps may be generated.

As related to step 205a, for each different protein-protein interaction map (and thus each different parameter setting), a set of predicted gene pathways are predicted. The predicted gene pathways are shown here as scored lists for purposes of illustration, though other data output forms (e.g., histograms) may additionally or alternatively be used. As related to step 205b, for each different protein-protein interaction map (and thus each different parameter setting), a set of pain phenotype associations are generated. The pain phenotype associations are shown here as radar plots for purposes of illustration, though other data output forms (e.g., histograms) may additionally or alternatively be used.

As related to step 208, the sets of pain phenotype associations from each different parameter setting are combined/overlayed, and as related to step 210, those phenotypes having sufficient overlap across the combined sets are identified as potential phenotypes for which the selected therapeutic compound can be targeted. The combined sets pain phenotype associations are shown here as an overlay chart/Venn diagram for purposes of illustration, though other data output forms may additionally or alternatively be used.

The use of multiple different parameter settings to generate multiple sets of pain phenotype associations in combination with subsequent combining/overlaying to select those with the most overlap provides benefits to the method. For example, the use of multiple different parameter settings beneficially broadens and differentiates the mapping of potential pain phenotypes, while the subsequent combining and overlaying allows for filtering and narrowing of the results to those that have robust cross-parameter occurrences. In other words, the use of multiple different parameter settings casts a wide net in the mapping of possible pathways affected by the selected therapeutic compound, while the combining/overlaying operation ensures that the end outputs are focused on those phenotypes/conditions that are most likely to be associated with the same molecular pathways.

### EXAMPLES

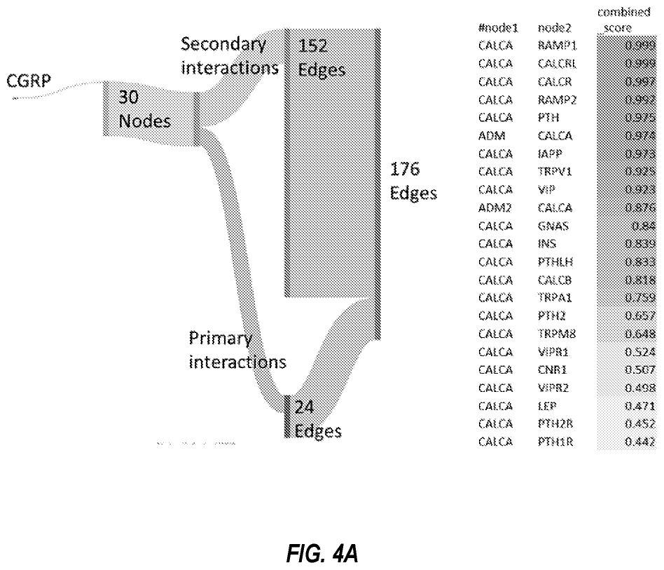

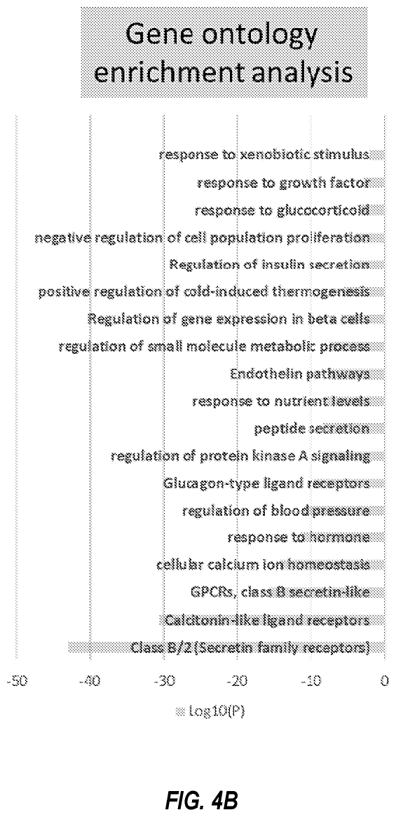

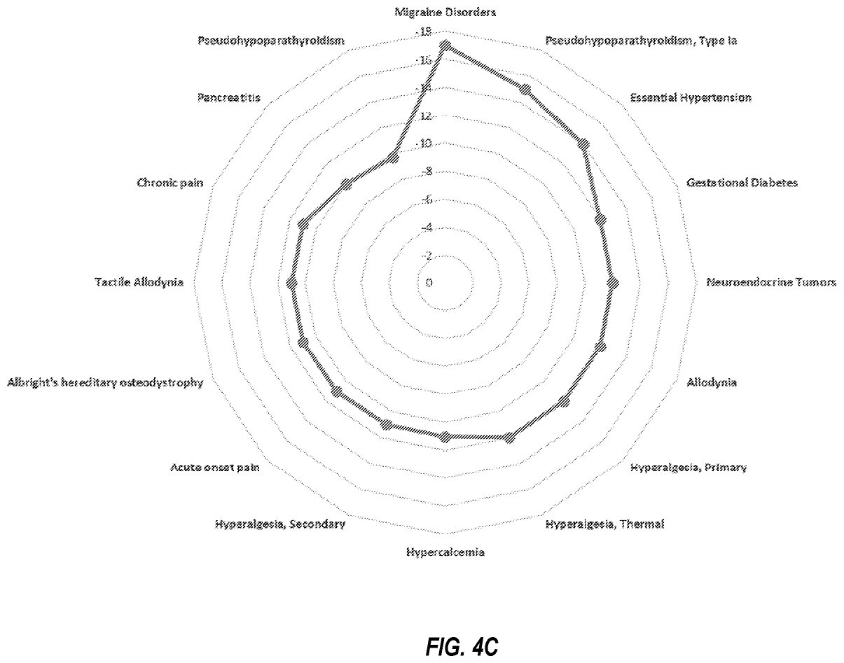

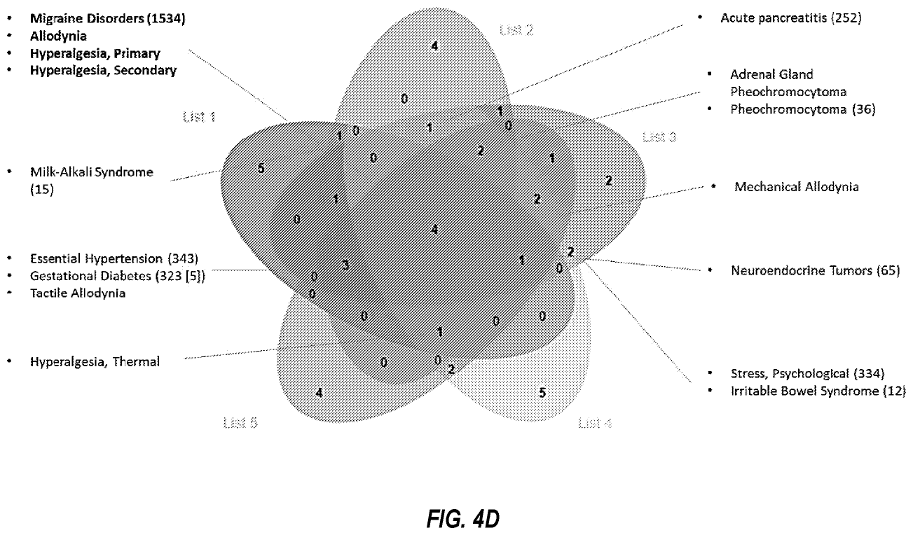

FIGS. 4A-4D illustrate example outputs using the method of FIG. 2 for the example therapeutic compound calcitonin gene-related peptide (CGRP). CGRP was selected as a test case because it known to be implicated in pain pathways and has known clinical efficacy alleviating migraines. The method should therefore be capable of generating output indicating similar relationships without requiring manual intervention.

FIG. 4A shows output from the protein-protein mapping operation of the method, FIG. 4B shows output from the gene ontology operation of the method, and FIG. 4C shows the pain phenotype association operation of the method, for a particular parameter setting. FIG. 4D illustrates the combined/overlayed results of pain phenotype associations from multiple parameter settings (each “list” show corresponds to the results from a different parameter setting).

The results show that the pain phenotypes with the most overlap include migraine disorders, allodynia, and hyperalgesia, which corresponds very well to the already known clinical properties of CGRP. The test case thus demonstrated that the analgesics platform described herein can effectively identify pain phenotypes/conditions for potential treatment using the selected therapeutic compound, without requiring any manual intervention to direct the algorithm toward the previously known result. This therefore illustrates the potential for the platform to identify new analgesic therapies that have yet to be discovered.

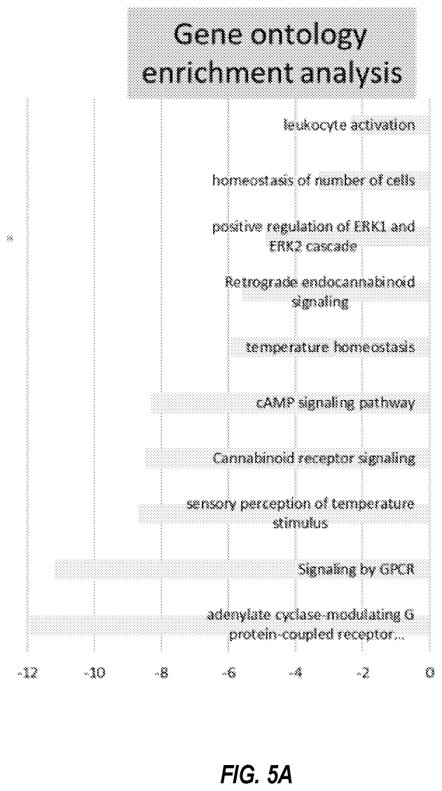

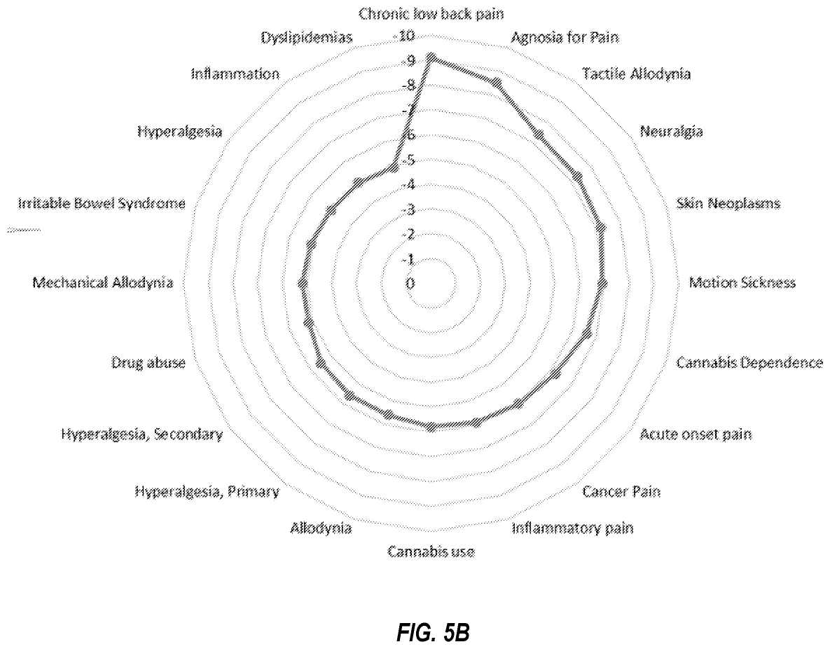

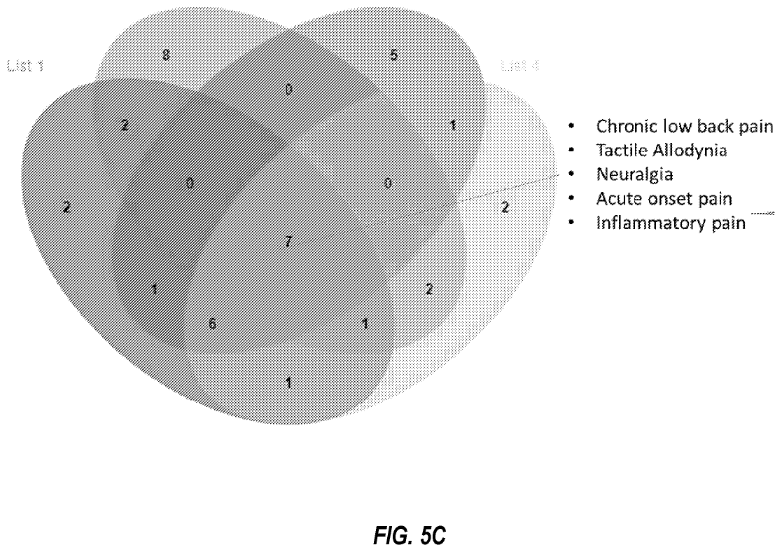

FIGS. 5A-5C illustrate example outputs using the method of FIG. 2 for the example compound GPR18. This test was similar to the CGRP test described above. However, unlike CGRP, GPR18 does not have any demonstrated clinical analgesic efficacy against any specified conditions. FIG. 5A shows output from the gene ontology operation, and FIG. 5B shows output from the pain condition association operation, for a particular parameter setting. FIG. 5C shows the combined/overlayed results of pain phenotype associations from multiple parameter settings (each “list” show corresponds to the results from a different parameter setting).

The results show that the set of pain phenotypes with the most overlap include chronic low back pain, tactile allodynia, neuralgia, acute onset pain, and inflammatory pain. One example pain condition associated with these phenotypes is radiculopathy. The platform thus demonstrates that GPR18 is a potential analgesic for treating radiculopathy.

A similar process was carried out using calcitriol (the active form of vitamin D) as the selected therapeutic compound. The results indicated calcitriol as a potential analgesic for use in treating keratin disorders such as pachyonychia congenita (a rare, autosomal dominant keratin disorder that causes painful keratoderma), Olmsted syndrome (another congenital keratin disorder characterized by painful, itchy palmoplantar keratoderma), and complex regional pain syndrome.

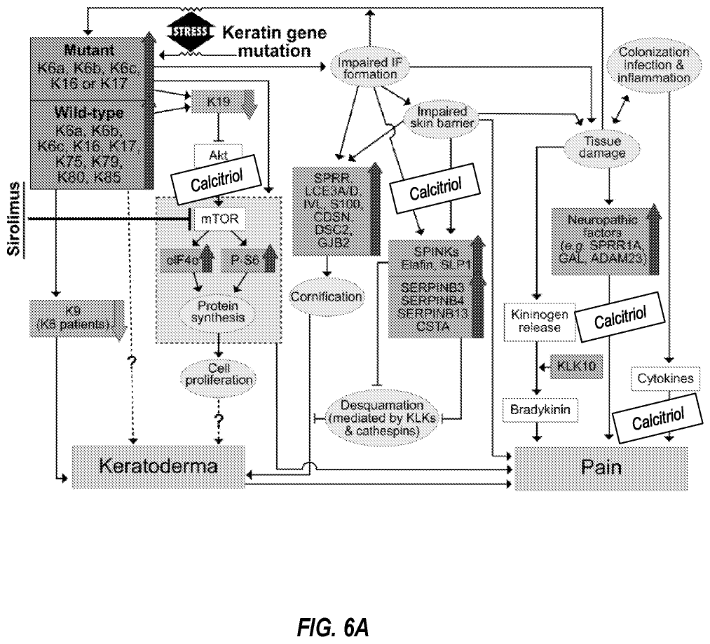

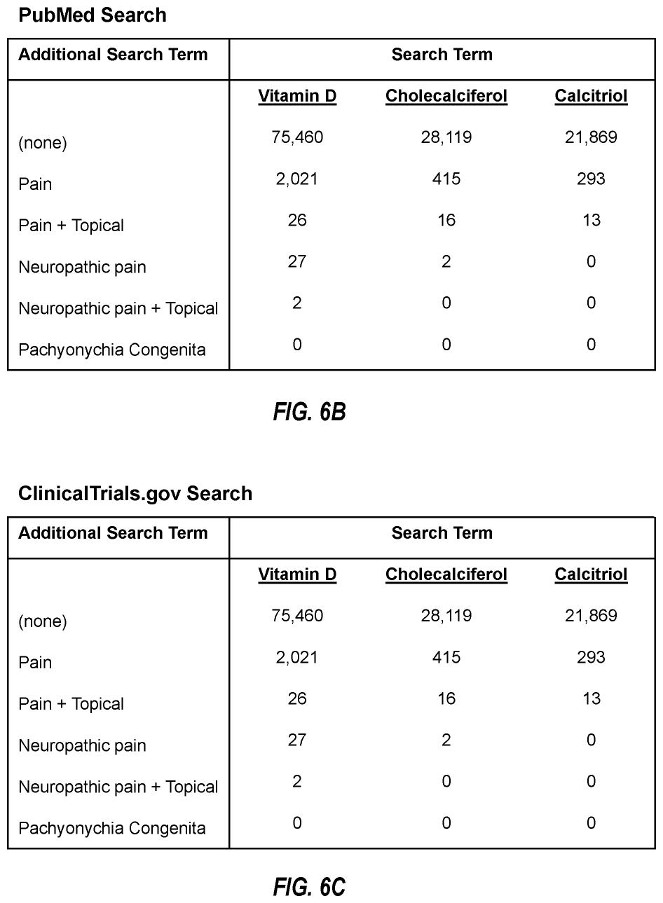

Calcitriol can interact with pachyonychia congenita pathways associated with mTOR, cytokines (e.g., TGF-b, IL1, IL8, IL10), as well as reduce neuropathic factors and promote repair of the skin barrier. FIG. 6A is a model of the molecular pathways involved in pachyonychia congenita (see Cao et al., Gene Expression Profiling in Pachyonychia Congenita Skin, J Dermatol Sci. 2015 March; 77 (3): 156-165) revised to show possible interaction points at which calcitriol can interact.

FIGS. 6B and 6C are charts summarizing the results of searches (PubMed and ClinicalTrials.gov, respectively) linking vitamin D, cholecalciferol, and/or calcitriol to specific disease conditions. These results indicate that little to no research has been conducted in investigating the relationship between these compounds and pain, particularly neuropathic pain. The presently described platform can thus function to provide paths for investigating novel analgesic therapies.

With respect to Olmsted syndrome, calcitriol may function to interact with TRPV3 associated pathways at EGFR, TGF, and NFkB (PGE2, IL1, NO, TGF-b). With respect to complex regional pain syndrome, calcitriol may function to modulate TNFa and IL6 cytokines, can normalize Mast cell function, reduce keratinocyte proliferation, and normalize sensory neurons via TRPV1 and EGFR.

### Additional Computer System Details

In this description and in the claims, the term “computer system” and similar terms is defined broadly as including any device or system—or combination thereof—that includes at least one physical and tangible processor and a physical and tangible memory capable of having stored thereon computer-executable instructions that may be executed by a processor. By way of example, not limitation, the term “computer system” or “computing system,” as used herein is intended to include personal computers, desktop computers, laptop computers, tablets, hand-held devices (e.g., mobile telephones, PDAs, pagers), microprocessor-based or programmable consumer electronics, minicomputers, mainframe computers, multi-processor systems, network PCs, distributed computing systems, datacenters, message processors, routers, and switches.

The memory may take any form and may depend on the nature and form of the computing system. The memory can be physical system memory, which includes volatile memory, non-volatile memory, or some combination of the two. The term “memory” may also be used herein to refer to non-volatile mass storage such as physical storage media, which can also be referred to as hardware storage devices.

The computing system also has thereon multiple structures often referred to as an “executable component.” For instance, the memory of computing system can include an executable component for operating the controller and/or functions of the elevation systems and/or circular reciprocation systems disclosed herein. The term “executable component” is the name for a structure that is well understood to one of ordinary skill in the art in the field of computing as being a structure that can be software, hardware, or a combination thereof.

For instance, when implemented in software, one of ordinary skill in the art would understand that the structure of an executable component may include software objects, routines, methods, and so forth, that may be executed by one or more processors on the computing system, whether such an executable component exists in the heap of a computing system, or whether the executable component exists on computer-readable storage media. The structure of the executable component exists on a computer-readable medium in such a form that it is operable, when executed by one or more processors of the computing system, to cause the computing system to perform one or more functions, such as the functions and methods described herein. Such a structure may be computer-readable directly by a processor—as is the case if the executable component were binary. Alternatively, the structure may be structured to be interpretable and/or compiled—whether in a single stage or in multiple stages—so as to generate such binary that is directly interpretable by a processor.

The term “executable component” is also well understood by one of ordinary skill as including structures that are implemented exclusively or near-exclusively in hardware logic components, such as within a field programmable gate array (FPGA), an application specific integrated circuit (ASIC), Program-specific Standard Products (ASSPs), System-on-a-chip systems (SOCs), Complex Programmable Logic Devices (CPLDs), or any other specialized circuit. Accordingly, the term “executable component” is a term for a structure that is well understood by those of ordinary skill in the art of computing, whether implemented in software, hardware, or a combination thereof.

The terms “component,” “service,” “engine,” “module,” “control,” “generator,” or the like may also be used in this description. As used in this description and in this case, these terms-whether expressed with or without a modifying clause—are also intended to be synonymous with the term “executable component” and thus also have a structure that is well understood by those of ordinary skill in the art of computing.

While not all computing systems require a user interface, in some embodiments a computing system includes a user interface for use in communicating information from/to a user. For example, a user interface can be used by a user to dictate their desired operation of the modified magnet assembly. The user interface may include output mechanisms as well as input mechanisms (e.g., I/O Devices). The principles described herein are not limited to the precise output mechanisms or input mechanisms as such will depend on the nature of the device. However, output mechanisms might include, for instance, speakers, displays, tactile output, projections, holograms, and so forth. Examples of input mechanisms might include, for instance, microphones, touchscreens, projections, holograms, cameras, keyboards, stylus, mouse, or other pointer input, sensors of any type, and so forth.

Accordingly, embodiments described herein may comprise or utilize a special purpose or general-purpose computing system. Embodiments described herein also include physical and other computer-readable media for carrying or storing computer-executable instructions and/or data structures. Such computer-readable media can be any available media that can be accessed by a general purpose or special purpose computing system. Computer-readable media that store computer-executable instructions are physical storage media. Computer-readable media that carry computer-executable instructions are transmission media. Thus, by way of example—not limitation—embodiments disclosed or envisioned herein can comprise at least two distinctly different kinds of computer-readable media: storage media and transmission media.

Computer-readable storage media include RAM, ROM, EEPROM, solid state drives (“SSDs”), flash memory, phase-change memory (“PCM”), CD-ROM or other optical disk storage, magnetic disk storage or other magnetic storage devices, or any other physical and tangible storage medium that can be used to store desired program code in the form of computer-executable instructions or data structures and that can be accessed and executed by a general purpose or special purpose computing system to implement the disclosed functionality of the invention. For example, computer-executable instructions may be embodied on one or more computer-readable storage media to form a computer program product. For the absence of doubt, such computer-readable storage media can also be termed “hardware storage devices,” which are physical storage media—not transmission media.

Transmission media can include a network and/or data links that can be used to carry desired program code in the form of computer-executable instructions or data structures and that can be accessed and executed by a general purpose or special purpose computing system. Combinations of the above should also be included within the scope of computer-readable media.

Further, upon reaching various computing system components, program code in the form of computer-executable instructions or data structures can be transferred automatically from transmission media to storage media (or vice versa). For example, computer-executable instructions or data structures received over a network or data link can be buffered in RAM within a network interface module (e.g., a “NIC”) and then eventually transferred to computing system RAM and/or to less volatile storage media at a computing system. Thus, it should be understood that storage media can be included in computing system components that also—or even primarily—utilize transmission media.

Those skilled in the art will further appreciate that a computing system may also contain communication channels that allow the computing system to communicate with other computing systems over, for example, a network. Accordingly, the methods described herein may be practiced in network computing environments with many types of computing systems and computing system configurations. The disclosed methods may also be practiced in distributed system environments where local and/or remote computing systems, which are linked through a network (either by hardwired data links, wireless data links, or by a combination of hardwired and wireless data links), both perform tasks. In a distributed system environment, the processing, memory, and/or storage capability may be distributed as well.

### ADDITIONAL TERMS & DEFINITIONS

Although particular examples of treatment compositions and methods are described herein, the examples do not limit the scope of the present disclosure. For example, where specific topical administration forms are described by way of example, it will be understood that other compositions and methods suitable for delivering calcitriol to dermal tissue may additionally or alternatively be used.

Unless otherwise indicated, numbers expressing quantities, proportions, percentages, or other measurements used in the specification and claims are to be understood as optionally being modified by the term “about” or its synonyms, even if the term does not expressly appear. Any numerical range recited herein is intended to include all subranges subsumed therein. When ranges are given, any endpoints of those ranges and/or numbers within those ranges can be combined within the scope of the present disclosure. Plural use of terms encompasses singular use of the terms and vice versa.

When the term “about,” “approximately,” “substantially,” “essentially,” or the like are used in conjunction with a stated amount, value, or condition, it may be taken to mean an amount, value, or condition that deviates by 10% or less, 5% or less, 1% or less, 0.1% or less, or 0.01% or less from the stated amount, value, or condition.

Any headings and subheadings used herein are for organizational purposes only and are not meant to be used to limit the scope of the description or the claims.

1. A system configured to link a therapeutic compound to one or more pain phenotypes or conditions, the system comprising:
one or more processors; and
one or more hardware storage devices having stored thereon computer-executable instructions which are executable by the one or more processors to cause the system to at least:
receive a selected therapeutic compound;
generate a protein-protein interaction map for each of one or more parameter settings for the selected therapeutic compound;
for each parameter setting generate a set of predicted gene pathways based on the corresponding protein-protein interaction map, and
generate a set of pain phenotype associations based on the protein-protein interaction map and the set of predicted gene pathways;
combine the sets of pain phenotype associations from each parameter setting;
identify one or more pain phenotypes having sufficient overlap across the combined sets of pain phenotypes associations as potential treatment targets; and
identify the selected therapeutic compound as a potential analgesic for the one or more potential treatment targets.

2. The system of claim 1, wherein the computer-executable instructions which are executable by the one or more processors further cause the system to generate patient selection criteria for a clinical trial related to the selected therapeutic compound and the one or more identified pain phenotype.

3. The system of claim 1, wherein the computer-executable instructions which are executable by the one or more processors further cause the system to identify a subset of patients that would be suitable for a clinical trial related to the selected therapeutic compound and the one or more identified pain phenotypes.

4. The system of claim 3, wherein the one or more identified pain phenotypes are compared against patient information to identify the subset of patients.

5. The system of claim 4, wherein the patient information is stored in a patient information database.

6. The system of claim 1, wherein the computer-executable instructions which are executable by the one or more processors cause the system to generate a protein-protein interaction map for each of a plurality of parameter settings for the selected therapeutic compound.

7. The system of claim 6, wherein the plurality of parameter settings comprises at least four different parameter settings.

8. The system of claim 1, wherein sufficient overlap is determined according to a pre-defined overlap threshold.

9. The system of claim 8, wherein the overlap threshold represents a minimum percentage of the sets of pain phenotype associations in which the identified pain phenotype must be present.

10. A computer-implemented method for linking a therapeutic compound to one or more pain phenotypes or conditions, the method comprising:
receiving a selected therapeutic compound;
generating a protein-protein interaction map for each of one or more parameter settings for the selected therapeutic compound;
for each parameter setting
generating a set of predicted gene pathways based on the corresponding protein-protein interaction map, and
generating a set of pain phenotype associations based on the protein-protein interaction map and the set of predicted gene pathways;
combining the sets of pain phenotype associations from each parameter setting;
identifying one or more pain phenotypes having sufficient overlap across the combined sets of pain phenotype associations as potential treatment targets; and
identifying the selected therapeutic compound as a potential analgesic for the one or more potential treatment targets.

11. The method of claim 10, further comprising generating patient selection criteria for a clinical trial related to the selected therapeutic compound and the one or more identified pain phenotypes.

12. The method of claim 10, further comprising identifying a subset of patients that would be suitable for a clinical trial related to the selected therapeutic compound and the one or more identified pain phenotypes.

13. The method of claim 12, wherein the one or more identified pain phenotypes are compared against patient information to identify the subset of patients.

14. The method of claim 13, wherein the patient information is stored in a patient information database.

15. The method of claim 10, further comprising generating a protein-protein interaction map for each of a plurality of parameter settings for the selected therapeutic compound.

16. The method of claim 15, wherein the plurality of parameter settings comprises at least four different parameter settings.

17. The method of claim 10, wherein sufficient overlap is determined according to a pre-defined overlap threshold.

18. The method of claim 17, wherein the overlap threshold represents a minimum percentage of the sets of pain phenotype associations in which the identified pain phenotype must be present.

19. A hardware storage product having stored thereon computer-executable instructions which are executable by a computer system to cause the computer system to execute a method as in claim 10.
